(19) United States
(12) Patent Application Publication
(10) Pub. No.: US 2007/0203145 A1
(43) Pub. Date: Aug. 30, 2007

(54) ZOPICLONE RESOLUTION

(76) Inventor: Jie Zhu, Nijmegen (NL)

Correspondence Address: SYNTHON IP INC, 7130 HERITAGE VILLAGE PLAZA, STE 202, GAINESVILLE, VA, 20155, US

(21) Appl. No.: 11/670,657

(22) Filed: Feb. 2, 2007

Related U.S. Application Data
(60) Provisional application No. 60/764,927, filed on Feb. 3, 2006.

(51) Int. Cl. A61K 31/498; C07D 487/02

(52) U.S. Cl. 514/249; 544/350

(57) ABSTRACT

Zopiclone can be effectively resolved, and the S-zopiclone isolated, by the use of L-tartaric acid, D-tartaric acid, (+)-ditoluoyltartaric acid, or (−)-ditoluoyltartaric acid.

The present application claims the benefit of priority under 35 U.S.C. §119(e) from U.S. Provisional Application Ser. No. 60/764,927, filed Feb. 3, 2006, the entire contents of which are incorporated herein by reference.

### BACKGROUND OF THE INVENTION

Zopiclone is the common name for 6-(5-chloro-2-pyridyl)-5-[(4-methyl-1-piperazinyl)carbonyloxy]-7-oxo-6,7-dihydro-5H-pyrrolo[3,4-b]pyrazine, which can be represented by the following formula (1).

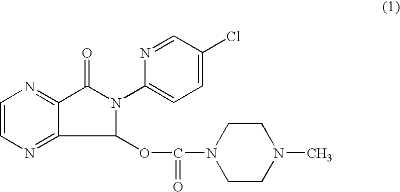

Zopiclone itself is a well known pharmaceutically active compound that is useful as a rapid acting hypnotic and which has been marketed in various countries since 1992 for treatment of insomnia. Zopiclone was disclosed in U.S. Pat. No. 3,862,149 and its priority application FR 72/00,505. It is apparent from the formula that zopiclone is a racemic compound, having one chiral carbon.

More recently the S-enantiomer of zopiclone has received attention. For example, U.S. Pat. No. 6,444,673 purports that S-zopiclone, referred to therein as the dextrorotary isomer, has twice the activity of the racemic mixture and less toxicity. The S-zopiclone isomer, which is also called “eszopiclone,” can be represented by the following formula (1A). 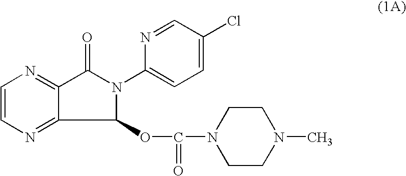
U.S. Pat. No. 6,444,673 indicates that racemic zopiclone can be resolved into substantially pure eszopiclone by a variety of known techniques including the use of an optically active salt. The only optically active acid specifically mentioned and exemplified is D(+)-O,O′-dibenzoyltartaric acid (also known as DBTA). The exemplified process is somewhat complicated, involving at least three crystallizations, and providing a relatively low yield of less than 25%. Similarly, see also WO92/12980.

WO00/69442 relates to desmethylzopiclone and the optical isomers thereof. One route described for making the enantiomers of desmethylzopiclone involves resolving zopiclone and then converting the optically pure zopiclone into the optically pure desmethylzopiclone. An example uses D(+)malic acid for resolving zopiclone and the specification further identifies L-N-benzyloxycarbonylphenylalanine, mandelic acid and dibenzoyltartaric acid as being useful. The authors also speculate that higher than molar amounts of the chiral acid should be used for the success of the resolution process. However, the published results of the resolution of zopiclone using malic acid are apparently not reproducible.

Given that eszopiclone has now been approved for marketing in the US (e.g., LUNESTA® by Sepracor Inc.), it would be desirable to find a more suitable process for the resolution of zopiclone. In particular, it would be desirable to have a process that could use an inexpensive resolution agent and/or provide a simple process without the need for extensive re-crystallizations or purifications.

### SUMMARY OF THE INVENTION

The present invention is based on the discovery that certain chiral acids, particularly a chiral tartaric acid, especially L-tartaric acid, can be a surprisingly effective resolution agent for zopiclone. Accordingly a first aspect of the invention relates to an S-zopiclone-L-tartrate compound. The compound is typically, though not necessary, crystalline. In a preferred aspect, the compound is an S-zopiclone-L-tartrate acetonitrile solvate. The compound can be readily separated from the corresponding R-zopiclone-L-tartrate compound by precipitation/crystallization/etc., and is preferably substantially free from the R-zopiclone moiety.

Another aspect of the invention relates to a process, which comprises reacting a mixture of R- and S-zopiclone and/or a salt thereof with L-tartaric acid in a solvent to form a solution containing zopiclone ions and L-tartaric acid ions. This solution, which is generally referred to as a solution of zopiclone-L-tartrate, can be used for resolving the zopiclone enantiomers. Generally, 0.4 to 1.2 molar equivalents of the tartaric acid in respect to zopiclone are used, more preferably 0.4 to 0.6 molar equivalents. In another preferred embodiment, 0.4-0.6 equivalents of tartaric acid together with 0.4-0.6 equivalents of another acid, which is an achiral acid, for instance hydrochloric acid, are used. Preferably the solvent comprises acetonitrile and optionally other co-solvents. The S-zopiclone can be separated by selectively precipitating an S-zopiclone-L-tartrate compound. The precipitation is selective in that more of the S-zopiclone compound is precipitated than the R-zopiclone compound; i.e., selective in the sense that one isomer is favored over the other even though both may be co-precipitated. The selective precipitation preferably provides a precipitate wherein at least 80%, preferably at least 85%, more preferably at least 90%, and still more preferably at least 95% of the zopiclone moieties contained therein are the S-zopiclone moiety, even if the starting zopiclone was racemic or otherwise about 50:50 R:S. Once an S-zopiclone enriched precipitate is obtained, it can be treated with base to form the corresponding enriched S-zopiclone. Typically when the neutralization results in precipitation of the S-zopiclone base, a further enrichment of the enantiomeric purity is also obtained. If further optical purity is desired, the whole process, or a portion thereof, can be repeated until the desired purity is obtained.

A further aspect of the present invention relates to a process for increasing the enantiomeric purity of S-zopiclone, which comprises:

(a) selectively precipitating from a solution, which contains zopiclone and tartaric acid as solutes and acetonitrile as a solvent, an S-zopiclone-L-tartrate acetonitrile solvate; and

(b) treating said S-zopiclone-L-tartrate acetonitrile solvate with a base to form enriched S-zopiclone.

Another aspect of the invention relates to a process for resolving R- and S-zopiclone using a chiral acid as a resolution agent, the improvement for which comprises using as said resolution agent a chiral acid selected from the group consisting of L-tartaric acid, D-tartaric acid, (+)-ditoluoyltartaric acid, and (−)-ditoluoyltartaric acid. Typically the process involves forming a chiral acid salt of enantiomerically impure zopiclone, selectively precipitating the R- or the S-zopiclone chiral acid salt to substantially separate (e.g. 80% enantiomeric purity or more) the R- and S-zopiclone chiral acid salts, and isolating the S-zopiclone as a free base from said chiral acid salt. Of the four chiral acids, L-tartaric acid is the most preferred.

### DETAILED DESCRIPTION OF THE INVENTION

The present invention is based on the discovery that certain chiral acid salts, particularly L-tartaric acid salts, of zopiclone are useful in easily separating the zopiclone enantiomers such as by fractional precipitation. In general, fractional precipitation comprises forming a salt of the corresponding zopiclone enantiomer and a chiral organic acid and selectively precipitating one of the enantiomeric forms. Such selective precipitation is based on the principle that salts consisting of a diastereomeric pair of chiral bases and chiral acids may have inherently different solubilities in certain solvents so that one diastereomeric pair preferably precipitates from the solvent, while the second pair preferably remains in the mother liquor (e.g. selective precipitation). Of course, there are no general rules for selecting a proper chiral acid or the proper precipitation conditions. Instead, the resolution agent and conditions are generally found, in each particular case, only by experimentation. Furthermore it is known that the precipitated salt product seldom consists of the pure enantiomer, and the success of the resolution may be evaluated by measuring the enantiomeric ratio or the enantiomeric enrichment in comparison with the starting values. Thus in some cases, the crystallization must be repeated several times, with each crystallization bringing greater and greater enantiomeric purity until the desired level is reached.

In the case of zopiclone, the previously disclosed processes for resolution proved to be not fully satisfactory. For example, the resolution of racemic zopiclone using the D(+)dibenzoyltartaric acid indeed requires the elaborate sequence of multiple crystallization to obtain a product (eszopiclone) of sufficient enantiomeric purity. But, the same is not true of the prior art D(+)malic acid process. Repetitions of this example, both with the same conditions and with modified conditions, were unable to achieve the stated results. To the contrary, the enantiomeric purity of the obtained product did not exceed the 55%, i.e. the precipitate (apparently zopiclone malate) still contained at least 45% of the undesired R-enantiomer. Obvious variations of the disclosed conditions did not bring any observable improvement of this value.

In searching for a better resolution agent, the following results were observed.

(+)-10-Camphorsulfonic acid, (+)-mandelic acid and (+)-aspartic acid did not give any positive results for the resolution, at least under the conditions that have been tested.

(−)-2,3:4,5-Di-O-isopropylidene-2-keto-L-gulonic acid does form an insoluble diastereomeric salt with zopiclone in a solvent system of ethanol/acetonitrile in a ratio of 2/1 (v/v). However, the precipitated product consists mainly of the undesired R-enantiomer, while the desired S-enantiomer maintains in the mother liquor and must be isolated therefrom within further production steps. It may be anticipated that the corresponding antipode, the (+)-2,3:4,5-Di-O-isopropylidene-2-keto-D-gulonic acid may produce the desired S-zopiclone directly under essentially the same conditions, however this compound is commercially not available.

As disclosed above, (+)-dibenzoyltartaric acid formed diastereomeric salt with zopiclone in a similar yield and enantiomeric enrichment as described in the prior art patent.

Quite surprisingly, (+)-ditoluoyltartaric acid was found as even more effective than (+)-dibenzoyltartaric acid, as it also formed a solid diastereomeric salt of S-zopiclone in a solvent system of ethanol/acetonitrile 2/1 (v/v), and the resolution was greater. Conventional optimization of the resolution using (+)-ditoluoyltartaric acid revealed that a diastereomeric salt with zopiclone can be obtained as a solid in about 44% yield, the solid product having the S:R diastereomeric ratio of 93:7 after the precipitation. After one re-crystallization and releasing the acid from the salt, the optical purity of obtained eszopiclone base can be easily brought to more than 99%, i.e. substantially free of the R-enantiomer. Similarly, the R-enantiomer of zopiclone may be obtained by essentially the same way using the (−)-ditoluyltartaric acid and the desired S-enantiomer may be isolated from the mother liquor in the essentially the same purity. The (+)-ditoluyltartaric acid is therefore a more effective resolution agent than the previously best known one, the (+)-dibenzoyltartaric acid, and thus forms one aspect of the present invention. The solvent is typically an acetonitrile-containing solvent as described hereinafter. However, (+)-ditoluoyltartaric acid is quite expensive and thus the overall cost of the process, despite its efficacy, can be high.

Quite surprisingly, it was further discovered that the relatively inexpensive and commercially available L-tartaric acid can provide for even better results. The tartrate salt of zopiclone is, in general, insoluble in the most common organic solvents, while it is well soluble in water. Further, the difference in solubilities between the salts with R- and S-enantiomers, respectively, is sufficiently high in typical solvent systems to provide enantiomerically well enriched salt product.

In particular, the invention provides for the precipitation of an (S)-zopiclone-L-tartrate compound of the following formula:

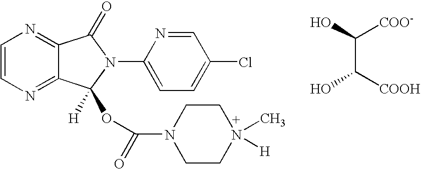

and preferably an (S)-zopiclone-L-tartrate monoacetonitrile solvate of the formula 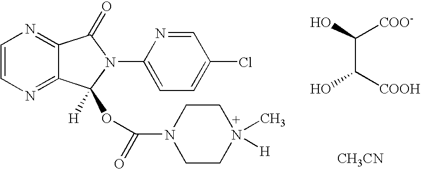
In general, the suitable solvent system for resolution of zopiclone using the L-tartaric acid comprises acetonitrile and/or the mixtures of acetonitrile with 1-90% of an organic co-solvent comprising at least one compound selected from an aliphatic C1-C4 alcohol (e.g. methanol or isopropanol), aliphatic C2-C6 ketone (e.g. acetone), aliphatic C1-C4 chlorinated hydrocarbon (chloroform or dichloromethane), aliphatic C2-C6 ester (e.g. ethyl acetate) and mixtures thereof. Preferred system comprises acetonitrile or a mixture of acetonitrile with ethanol.

The process of the invention may be schematically depicted as follows:

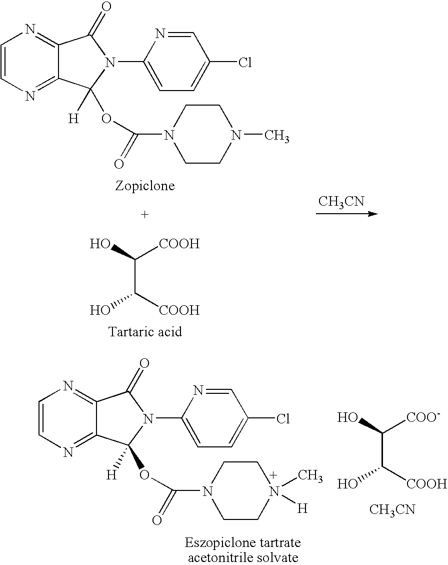

In a first suitable arrangement of resolution, an essentially 1:1 molar mixture of racemic zopiclone and L-tartaric acid (in practice, a 1:0.8 to 1:1.2 molar ratio may be used) is refluxed in the above acetonitrile-based solvent system, e.g. in acetonitrile or in the acetonitrile/ethanol system, and cooled to room temperature. The process of heating and cooling may be repeated. After stirring the slurry for a certain time (to reach an equilibrium), the precipitated solid comprising mostly the salt of L-tartaric acid with the S-enantiomer of zopiclone is separated by filtration and optionally washed and dried. In general, the enantiomeric purity of the precipitated product is at least 80%, typically at least about 85%, preferably at least 90%, and more preferably at least 95%, in the solvent system containing acetonitrile.

In a second and advantageous mode of resolution, it is even possible to use only half a molar equivalent (0.4-0.6 molar equivalent, preferably 0.5) of the L-tartaric acid in respect to zopiclone. In the above solvent system comprising acetonitrile, the S-enantiomer precipitates as the tartrate salt (essentially 1:1 molar ratio between zopiclone and tartaric acid moieties), while the R-enantiomer maintains in the solution as the free base. In particular, the resolution is very efficient when using acetonitrile/dichloromethane solvent system, in which the differences of solubilities between S-zopiclone L-tartrate and the R-zopiclone base are enormous.

Furthermore, in a third resolution mode, it is also possible to use the half molar equivalent of the L-tartaric acid together with a half molar equivalent (0.4-0.6 molar equivalent) of a second acid, which is an achiral acid. Typically the second acid is hydrochloric acid. Then, the resolution is based on the big difference in solubilities between the insoluble (S)-zopiclone L-tartrate and the soluble (R)-zopiclone salt of the second acid, typically the hydrochloride.

The possibility of using only a half molar equivalent of tartaric acid is surprising in light of the teaching of the prior art, as conventionally a molar equivalent of the resolution acid should be used for the resolution of zopiclone. The use of the half molar equivalent has not only a considerable economic impact but also a practical one. For example, it is generally not necessary to heat and cool the reaction mixture to induce precipitation, but the whole resolution process may even be performed at ambient temperature, under spontaneous precipitation of the tartrate salt of the desired S-enantiomer of zopiclone.

In general, however, it is not excluded that the precipitation is induced by seeding, cooling the reaction mixture, decreasing the volume of the solvent system and/or by adding a contrasolvent in any of the above three precipitation modes disclosed above and, as is well known, such techniques may increase the yield or efficiency of the precipitation.

Acetonitrile is a preferred solvent in the resolution process of the present invention. It should be noted that crystalline zopiclone tartrate salt may also be prepared by treating zopiclone with L-tartaric acid in a solvent which does not comprise acetonitrile (e.g. in methanol, isopropanol or acetone). However, it is difficult to achieve significant preferential precipitation in such systems. Typically only negligible enantiomeric enrichment results. But if the zopiclone-L-tartrate is then recrystallized from an acetonitrile-comprising solvent, a resolution is performed, and the precipitated product is substantially enriched by the corresponding (S)-enantiomer.

Thus, in a fourth mode of resolution, racemic or substantially racemic zopiclone L-tartrate, whenever prepared (e.g. it may be prepared within a synthetic process yielding zopiclone molecule or it may be prepared by treating the produced zopiclone with L-tartaric acid in a non-acetonitrile solvent) may be resolved into enantiomers by recrystallization from an acetonitrile-comprising solvent.

In analysing the structure of the precipitated solid product consisting essentially of the zopiclone L-tartrate and being obtained by any of the above four modifications of the resolution procedure (even regardless of whether it is enriched by the corresponding (S)-enantiomer or not), it has been found that the ratio between zopiclone and tartrate moiety is essentially 1:1 (analytical values may vary from 1:0.8 to 1:1.2). Thus, the proper denomination of the isolated product should be zopiclone (1:1) tartrate, or zopiclone hydrogentartrate, or zopiclone monotartrate.

Furthermore, the isolated product obtained by precipitating from an acetonitrile-comprising solvent, filtering, and drying at ambient temperature, is often in the form of an acetonitrile solvate. Typically the solvate comprises about one molar equivalent of acetonitrile (analytical results may vary from 0.8 to 1.2 mol). Therefore, such precipitated product is considered a zopiclone tartrate monoacetonitrile solvate, comprising mostly the (S)-zopiclone L-tartrate monoacetonitrile solvate. Typically, the enantiomeric content of the (S)-zopiclone moiety in the product is higher than 85%, more typically higher than 95%, in respect to the overall zopiclone molecules present.

In general, it is not necessary to enhance the enantiomeric purity of the precipitated product in this stage. Should it be necessary, however, it is possible to recrystallize the product from the solvent system comprising acetonitrile, advantageously from the same solvent system as used for the resolution. A suitable enantiomeric purity of the salt product for use as an intermediate in an industrial process of making enantiomerically pure eszopiclone base is generally at least 95%.

The eszopiclone tartrate monoacetonitrile solvate is a stable product, which essentially does not release acetonitrile during conventional drying at temperatures below 100° C. However, it is possible to prepare an acetonitrile-free (desolvated) zopiclone tartrate, e.g., by intensive drying at diminished pressure at temperatures higher than 100 ° C. Such product consists essentially of only the zopiclone and tartrate moieties.

Both the zopiclone tartrate acetonitrile solvate and the desolvated zopiclone tartrate, typically comprising more than 95% of the desired S-enantiomer, are useful intermediates for making the zopiclone free base, typically the (S)-zopiclone free base (the eszopiclone). In a suitable process, the salt is dissolved in water, a molar equivalent of a suitable base is added (typically, an inorganic base such as sodium hydroxide may be used) and the formed free base of the zopiclone is extracted by an organic solvent immiscible with water, typically by dichloromethane. The solid base is obtained by decreasing the volume of the solution, typically by an evaporation of the solvent.

The crude eszopiclone product may be purified by a recrystallization from a suitable solvent. Typical solvent for recrystallization is acetonitrile.

In general, the starting enantiomeric purity of the product may be even enhanced within the isolation process. Typically, the salt product having the original purity of 95% may provide for enantiomerically 99% pure eszopiclone base after the isolation and one recrystallization.

By conventional fine tuning, it is expected that a resolution can be performed, by which the isolated solid tartrate salt of zopiclone may reach a 45% yield or more with diasteromeric purity of at least 95%, preferably at least 97%, without further re-crystallization. By releasing the L-tartaric acid, an optically pure eszopiclone may be obtained with enantiomeric purity higher than 99.9%. The overall yield of the resolution process is typically at least 30% and may reach 40% or more.

Mother liquors remaining after the resolution and isolation of the S-zopiclone tartrate are significantly enriched by the R-enantiomer of zopiclone. Depending on the process, it may be present essentially as zopiclone tartrate, zopiclone base or a salt of zopiclone with the second achiral acid. If desired, such product may be isolated from the mixture by obvious means. For instance, the mother liquor may be concentrated, the rest dissolved in water, basified, if necessary, to an alkaline pH and the free base of the zopiclone may be extracted by an organic solvent, e.g. by dichloromethane.

The process disclosed above may be also used for making the R-enantiomer of zopiclone, using the D(−)tartaric acid as the resolution agent and performing essentially the same procedure. An R-zopiclone-D-tartrate would be selectively precipitated while the S-zopiclone-D-tartrate in its dissociated form would preferentially remain in the solution. The S-enantiomer may be obtained from mother liquors after the resolution and isolated by obvious means.

The process disclosed above may be also use for the improvement of the enantiomeric purity of a product comprising the (S)-enantiomer of zopiclone in a mixture with an (R)-enantiomer. Such a product may be, for instance, a product of an unsuccessful resolution of the racemate, for instance when using the malic acid or the dibenzoyltartaric acid, or a product of an enantio selective synthesis yielding predominantly, but not solely one of the enantiomers of zopiclone. Therefore, the starting material of the above process is not limited to the racemic zopiclone, but any mixture of (S) and (R) enantiomers may be used as well.

The invention will be further described by the following non-limiting examples.

### EXAMPLES

### Example 1

### Zopiclone L-tartrate acetonitrile solvate

To a stirred mixture containing 19.4 g of racemic zopiclone, 500 ml of acetonitrile, 250 ml of ethanol and 50 ml of dichloromethane, it was added 4.0 g of L-tartaric acid. The flask was immersed into a pre-heated oil bath (˜100° C.). The mixture was refluxed for 15 minutes. Heating was stopped and the mixture was seeded and stirred for ˜20 hours. Solid was collected by filtration. 12.97 g crystalline solid was obtained after drying at on an open dish over night at room temperature. The enantiomeric purity of the solid was ˜97% (S-enantiomer) based on chiral HPLC analysis.

### Example 2

### Zopiclone L-tartrate acetonitrile solvate

To a stirred mixture containing 19.4 g of racemic zopiclone, 500 ml of acetonitrile, 250 ml of ethanol and 50 ml of dichloromethane, it was added 2.5 ml of HCl solution (5˜6 N) in 2-propanol. The mixture turned to a clear solution. Then, 4.0 g of L-tartaric acid was added. The flask was immersed into a pre-heated oil bath (˜100° C.). The mixture was refluxed for 15 minutes. Heating was stopped and the mixture was seeded and stirred for ˜20 hours. Solid was collected by filtration. 12.95 g crystalline solid was obtained after drying at on an open dish over night at room temperature. The enantiomeric purity of the solid was ˜96% (S-enantiomer) based on chiral HPLC analysis.

### Example 3

### Eszopiclone

To a stirred mixture of 12.46 g zopiclone L-tartrate monoacetonitrile solvate (96% of the S-enantiomer) in a mixed solvent consisting of 150 ml of water and 150 ml of dichloromethane, there was added, at room temperature, 2N aqueous solution of NaOH until the pH value reached to approx. 11. Both layers were separated. Dichloromethane layer was washed with water (2×50 ml). Then it was dried over Na₂SO₄ and concentrated to give a crude solid (8.12 g). The crude solid was re-dissolved in 100 ml of acetonitrile, with stirring and heating, to give a clear solution. Then it was cooled down to room temperature and stirred for 1 hour. Crystalline solid was collected by filtration and washed with acetonitrile (20 ml). 6.13 g of crystalline solid was obtained after drying at room temperature on an open dish overnight. The analysis showed an optical purity of 99.9%. Filtrate was partly concentrated to 25 ml to generate second crop of the crystalline product (1.53 g, after drying), which had an optical purity of 98.85%.

Each of the patents mentioned above are incorporated herein by reference. The invention having been described it will be obvious that the same may be varied in many ways and all such modifications are contemplated as being within the scope of the invention as defined by the following claims.

1. An S-zopiclone-L-tartrate compound.

2. The compound according to claim 1, which is substantially free from R-zopiclone and any R-zopiclone salt.

3. The compound according to claim 1, which is an S-zopiclone-L-tartrate monoacetonitrile solvate.

4. The compound according to claim 1, in crystalline form.

5. A process, which comprises:
reacting a mixture of R- and S-zopiclone and/or a salt thereof with L-tartaric acid in a solvent to form a solution containing zopiclone ions and L-tartaric acid ions; and
selectively precipitating an S-zopiclone-L-tartrate compound from said solution to form an S-zopiclone-moiety enriched precipitate.

6. The process according to claim 5, wherein said selective precipitation provides a precipitate wherein at least 80% of the zopiclone moieties contained therein are the S-zopiclone moiety.

7. The process according to claim 5, wherein said selective precipitation provides a precipitate wherein at least 95% of the zopiclone moieties contained therein are the S-zopiclone moiety.

8. The process according to claim 5, wherein said mixture of R- and S-zopiclone is about 50:50.

9. The process according to claim 5, wherein 0.4-1.2 molar equivalents of tartaric acid are provided for said reaction per 1 mole of zopiclone.

10. The process according to claim 5, wherein 0.4-0.6 molar equivalents of tartaric acid are provided for said reaction per 1 mole of zopiclone.

11. The process according to claim 5, wherein said solvent comprises acetonitrile.

12. The process according to claim 11, wherein the solvent further comprises an aliphatic C1-C4 alcohol, an aliphatic C2-C6 ketone, an aliphatic C1-C4 chlorinated hydrocarbon, and/or an aliphatic C2-C6 ester.

13. The process according to claim 11, wherein the solvent further comprises methanol, isopropanol, acetone, chloroform, dichloromethane or ethyl acetate.

14. The process according to claim 5, wherein said precipitation is spontaneous after reacting the zopiclone and tartaric acid in the solvent.

15. The process according to claim 5, wherein the precipitation is carried out with cooling, decreasing the amount of the solvent and/or by adding a contrasolvent.

16. The process according to claim 5, wherein the precipitation is carried out with seeding.

17. The process according to claim 5, wherein said solvent comprises acetonitrile and said precipitate contains S-zopiclone-L-tartrate monoacetonitrile solvate.

18. The process according to claim 17, which further comprises treating said precipitate with base and isolating enriched S-zopiclone.

19. The process according to claim 18, which further comprises recrystallizing said enriched S-zopiclone to obtain at least 99% enantiomerically pure S-zopiclone.

20. A process for increasing the enantiomeric purity of S-zopiclone, which comprises:
(a) selectively precipitating from a solution, which contains zopiclone and tartaric acid as solutes and acetonitrile as a solvent, an S-zopiclone-L-tartrate acetonitrile solvate; and
(b) treating said S-zopiclone-L-tartrate acetonitrile solvate with a base to form enriched S-zopiclone.

21. In a process for resolving R- and S-zopiclone using a chiral acid as a resolution agent, the improvement for which comprises using as said resolution agent a chiral acid selected from the group consisting of L-tartaric acid, D-tartaric acid, (+)-ditoluoyltartaric acid, and (−)-ditoluoyltartaric acid.

22. A process which comprises:
forming a chiral acid salt of enantiomerically impure zopiclone;
selectively precipitating the R- or the S-zopiclone chiral acid salt to substantially separate the R- and S-zopiclone chiral acid salts; and
isolating said S-zopiclone as a free base from said chiral acid salt;
wherein said chiral acid is selected from the group consisting of L-tartaric acid, D-tartaric acid, (+)-ditoluoyltartaric acid, and (−)-ditoluoyltartaric acid.

23. The process according to claim 22, wherein said selective precipitation is carried out in an acetonitrile-containing solvent.
